(19) United States
(12) Patent Application Publication
(10) Pub. No.: US 2006/0020021 A1
(43) Pub. Date: Jan. 26, 2006

(54) Use of compounds derived from 2,3-dehydronaringenin for the treatment of inflammatory processes and pharmaceutical composition containing said derivatives

(75) Inventors: Juan Lorente Salinas, Murcia (ES); Julian Castillo Sanchez, Murcia (ES); Obdulio Benavente-Garcia Garcia, Murcia (ES); Vicente Vicente Ortega, Murcia (ES); Josefa Yanez Gascon, Murcia (ES); Francisco Solano Munoz, Murcia (ES); Miguel Alcaraz Banos, Murcia (ES); Jose Carlos Garcia Borron, Murcia (ES); Jose Antonio Lozano Teruel, Molina de Segura (ES)

Correspondence Address: MERCHANT & GOULD PC, P.O. BOX 2903, MINNEAPOLIS, MN, 55402-0903, US

(73) Assignee: FURFURAL ESPANOL, S.A., Alcantarilla (ES)

(21) Appl. No.: 11/186,121

(22) Filed: Jul. 20, 2005

(30) Foreign Application Priority Data
Jul. 20, 2004 (EP) EP04380155.4

(51) Int. Cl. A61K 31/353

(52) U.S. Cl. 514/456

(57) ABSTRACT

The present invention refers to the use of 2,3-dehydronarinegenin (apigenin) derivatives of Formula (I) for the treatment or prophylaxis of inflammatory processes and chronic diseases derived from inflammatory processes, as well as to a pharmaceutical composition containing them, together with excipients.

### FIELD OF THE INVENTION

The present invention is encompassed within the field of drugs for the treatment of inflammatory processes; it specifically refers to the use of 2,3-dehydronaringenin (apigenin) derivatives, of Formula (I), for the treatment or prophylaxis of inflammatory processes and chronic diseases derived from inflammatory processes, as well as to a pharmaceutical composition containing them, together with excipients.

### STATE OF THE ART

One of the major health problems in the current society is related to the inflammatory processes and diseases derived therefrom due to the discomforts and pain they cause in the patient. A classic example of this type of diseases related to inflammatory processes is rheumatoid arthritis (hereinafter, RA). The treatment of rheumatoid arthritis with drugs includes two large groups of drugs: one of them encompasses those serving to relieve the pain and inflammation on a short-term basis. They are useful for decreasing the inflammation and overcoming the “day-to-day” pain, but they do not serve for modifying the evolution of the disease on a long-term basis. The so-called non-steroidal anti-inflammatory drugs (NSAIDs) and corticoids are included in this group.

NSAIDs are cyclooxygenase inhibitors. The most frequent side effects of NSAIDs, gastrointestinal side effects, are related to their acidic nature and to prostaglandin inhibition. They can be expressed by different lesions, ranging from gastritis to ulcers, preferably gastric. These lesions can be asymptomatic or can be manifested with microscopic bleeding or clinically evident digestive hemorrhage. Risk factors for showing these complications are an elderly age and a prior history of epigastralgia or peptic ulcer. It is recommendable to take a gastric cytoprotective agent in these patients, and it is necessary to observe the occurrence of unusual digestive discomforts or changes in the deposition suggestive of digestive bleeding.

NSAIDs can cause a creatinine clearance decrease, by affecting the vasodilator function of prostaglandins. This is particularly important in elderly patients and in those with previously damaged renal function. Patients can develop acute interstitial nephropathy, papillary necrosis, and rarely, nephrotic syndrome. Secondary cutaneous, hepatic, hematological or neurological effects are less frequent. The choice of a specific NSAID is empirical and must be based on the pharmacokinetics of each preparation, including its half-life and its toxicity, as well as the degree of pain or inflammation of the joint process. The possible interaction with other drugs which the patient may be taking must be taken into account. It is advisable to use a specific preparation during two weeks at suitable doses before considering it ineffective and changing to a different drug. The use of two different NSAIDs does not add efficacy to the treatment and implies a greater risk of side effects.

Since the introduction of cortisone for the treatment of RA almost 50 years ago, glucocorticoids have been widely used for said purpose. After the initial enthusiasm due to their beneficial effects, the use decreased due to the high frequency of important side effects and the lack of evidence that the disease went into remission with their use. Their use being restricted due to these reasons for years, they are currently used more frequently given their clear anti-inflammatory action and the evidence, according to recent studies, suggesting that their use at low doses decreases the occurrence of erosions. Their use in RA must be reserved for those patients in whom the short-term benefits are greater than the risks of their long-term use, and always as part of a programmed treatment. Glucocorticoids are currently being questioned due to the fact that they seem to be related to an increase in the incidence of cardiac diseases in patients treated with these drugs.

Another large group of anti-inflammatories integrated by drugs which do not serve for treating pain in a determined moment, but which act by making the long-term activity of the disease lower, are disease-modifying anti-rheumatic drugs, slow-action remission inducers, immunosuppressants and cytotoxics. The use of these drugs is empirical and although all of them have shown their efficacy in the treatment of RA, long-term studies show discouraging results with regard to their benefit/toxicity ratio. They take weeks and even months in taking effect. There is no unanimous criterion concerning which treatment regimen to use or which drug or drug combination to choose. They are not effective in 100% of the patients, thus it is usual that the physician has to sequentially prescribe several of them until finding which one is the most effective and best tolerated. In this group are methotrexate, gold salts, chloroquine, sulfasalazine, D-penicillamine, azathioprine, cyclosporine . . . etc. They generally require control by the rheumatologist and close collaboration of the patient. When a drug of this type is prescribed, in addition to the degree of activity of the disease, the possible results which are expected to be obtained and the potential toxic effects thereof must be taken into account.

Therefore, there is the need to find compounds with a good anti-inflammatory activity and which in turn have scarce or zero side effects due to their cytotoxicity, thus allowing for long-term treatments.

The use of polyphenolic substances of a natural origin has resulted in combining a low toxicity with interesting antioxidant and anti-inflammatory properties, in addition to being moderate cyclooxygenase inhibitors.

Specifically, the 2,3-DHNA (apigenin) glycosides present in numerous plants (and particularly in chamomile) have shown to be important anti-inflammatory agents of a natural origin. The effectiveness of chamomile in the relief of symptoms of gastritis, gastric ulcers and other inflammatory processes in mucosae is due to the anti-inflammatory properties which are provided thereto by the 2,3-DHNA glycosides it contains (Merfort et al., 1994).

Some recent studies have shown that 2,3-DHNA could be effective in the treatment of inflammatory processes in the skin induced by free radicals (such as UV, X or γ irradiation, or chemical agents). Intradermal applications of liposomal 2,3-dehydronaringenin-7-glycoside inhibit the cutaneous inflammation caused by xanthine oxidase and cumene hydroperoxide in a dose-dependent manner. These results are in accordance with the properties as in-vitro scavengers of peroxide radicals and of the 2,3-DHNA superoxide anion, and which indicate that their antioxidant properties contribute to their anti-inflammatory effect in model systems (Fuchts and Milbradt, 1993). Studies carried out about the penetration into the skin of 2,3-DHNA, luteolin and 2,3-dehydronaringenin-7-glycoside have concluded that these flavonoids are not only absorbed in the surface of the skin, but they also penetrate in the deep layers of the skin. This is very important for their topical application as an antiphlogistic agent (Mefort et al., 1994).

Although there are several publications disclosing the use of 2,3-DHNA (apigenin) in pharmaceutical compositions for the treatment of certain diseases such as colon cancer (Walle and Halushka, 2001), diabetes (Ziegler, 2000), hepatic cholestasis (Gebhardt, 2001) or as an anxiolytic (Cassels et al., 1999), there is no publication in which the effectiveness of the use of 2,3-DHNA for the treatment of chronic diseases of an inflammatory character has been disclosed.

### DESCRIPTION OF THE INVENTION

A group of compounds has now been found which solves the drawback of the lack of compounds with scarce or zero cytotoxicity, with a good anti-inflammatory activity in inflammatory processes and/or chronic diseases generated by them, which furthermore allows for a prolonged administration to the patient. Therefore, according to a first aspect, the present invention refers to the use of compounds of Formula (I) 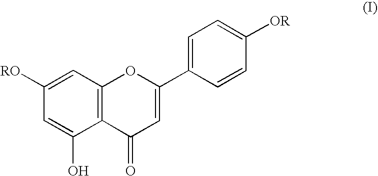
wherein the radicals R, independently from one another, are selected from among a hydrogen atom, a cation or a linear or branched C₁-C₃ alkyl group in the preparation of a drug for the treatment or prophylaxis of inflammatory processes and chronic diseases derived from inflammatory processes.

According to a preferred embodiment of the invention, in the compound of Formula (I), R is a cation of an alkaline metal. According to an even more preferred embodiment, in the compound of Formula (I), R is a potassium cation.

The compounds of Formula (I) have demonstrated a special efficacy in the treatment of rheumatoid arthritis and Chrom's disease. Therefore, according to a preferred embodiment of the invention, the latter refers to the use of compounds of Formula (I) in the preparation of a drug for the treatment of rheumatoid arthritis or Chrom's disease.

Likewise, the compounds of Formula (I) have shown a special efficacy in the treatment of inflammatory processes having the release of prostaglandins and thromboxanes as an origin. In this manner, another preferred embodiment of the invention is the use of compounds of Formula (I) in the preparation of a drug for the treatment of inflammatory processes having the release of prostaglandins and thromboxanes as an origin.

According to a second aspect, the present invention refers to a pharmaceutical composition comprising a compound of formula 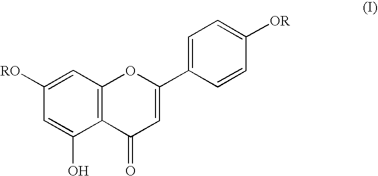
wherein the radicals R, independently from one another, are selected from among a hydrogen atom, a cation or a linear or branched C₁-C₃alkyl group, saline solution, a glycol selected from among the C₁-C₃ alcohols, an alkaline phosphate and an alkaline hydroxide.

Preferably, R is a cation of an alkaline metal; more preferably, R is a potassium cation.

According to a preferred embodiment, the glycol is propylene glycol.

According to another preferred embodiment, the alkaline phosphate is potassium phosphate.

Preferably, the alkaline hydroxide is potassium hydroxide.

Below, the invention will be additionally described in more detail by means of a series of examples and drawings, merely as an illustrative and by no means limiting example.

FIG. 1 shows a graph of the variation of the extent of the inflammation of a paw of a mouse over time, caused by intradermal injection of carragenin.

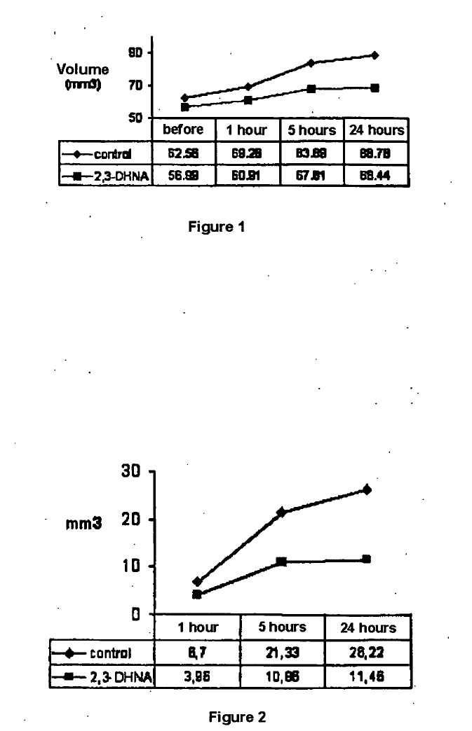

FIG. 2 shows a graph of the variation of the extent of the inflammation of a paw of a mouse over time, caused by the intraperitoneal administration of the compound of Formula (I).

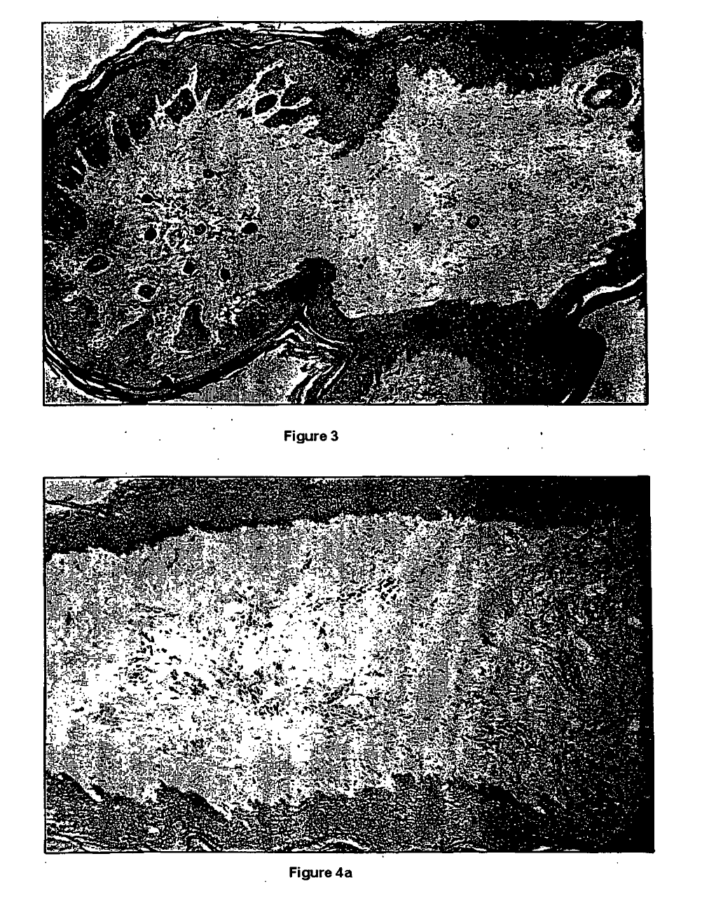

FIG. 3 shows a microscopic image of the subplantar region corresponding to degree 0 (HE 125X).

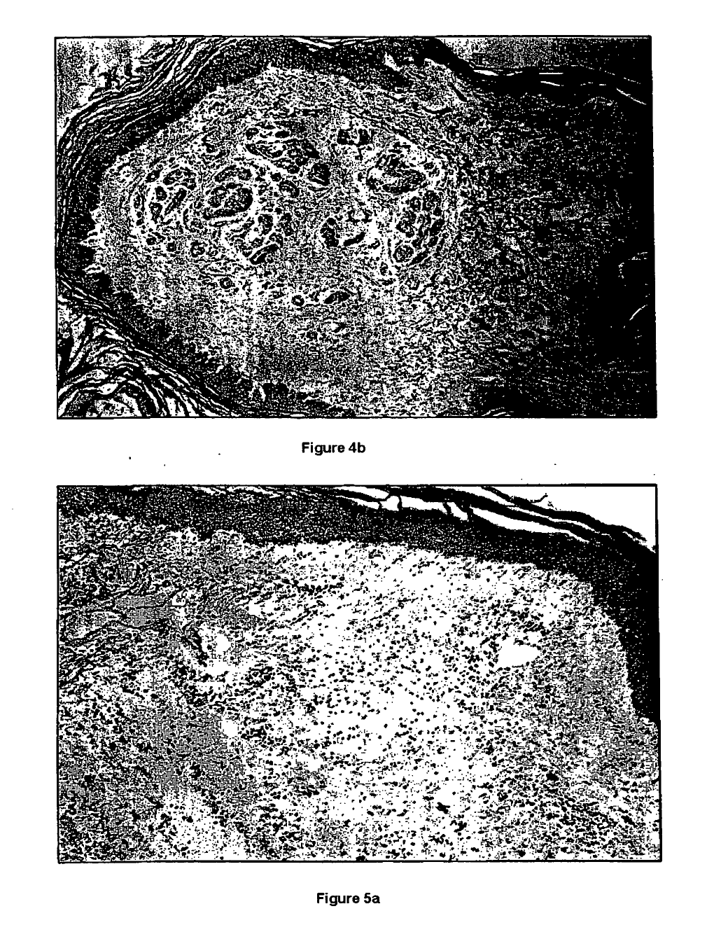

FIG. 4a shows a microscopic image of the subplantar region corresponding to inflammatory phenomena (degree 1) characterized by dilated vessels and congestives with extensive areas of edema and small accumulations of preferably perivascular polymorphonuclear infiltrates (HE 125X).

FIG. 4b shows a detail of the edematous phenomena and perivascular infiltrates (HE 200X).

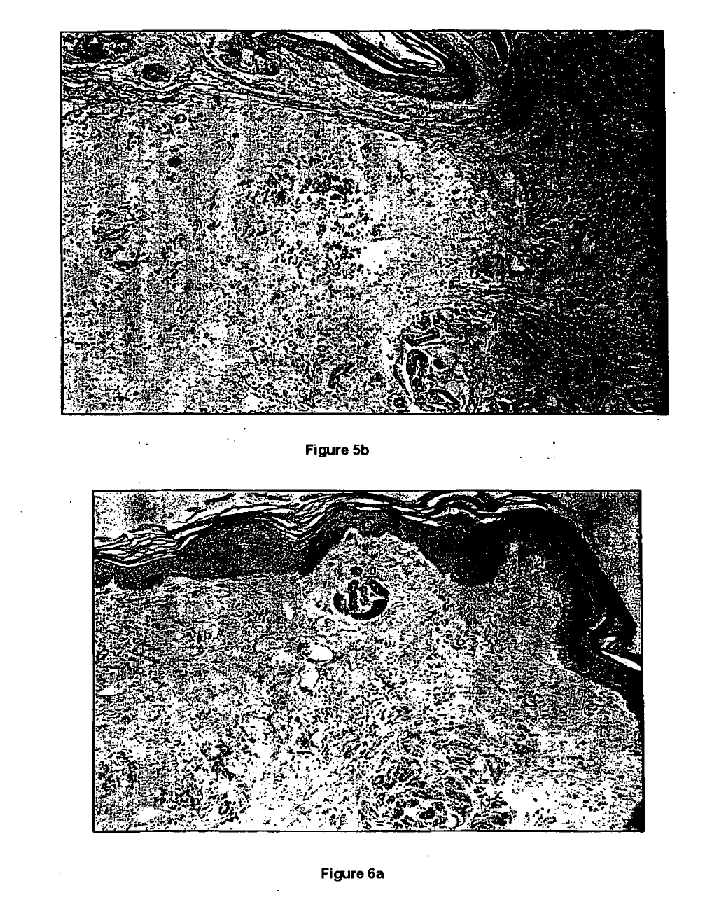

FIG. 5a shows a microscopic image with extensive areas of edema and numerous polymorphonuclear leukocyte infiltrates preferably arranged in a diffuse manner (HE 500X).

FIG. 5b shows a detail of the areas of edema occupying the entire image, as well as of the extensive polymorphonuclear infiltrates (HE 312′5X).

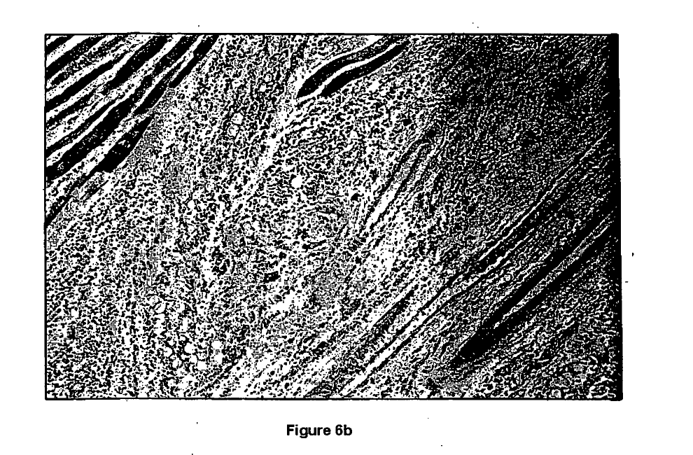

FIG. 6a shows a microscopic image of the subplantar cutaneous area with pronounced edematous phenomena and predominance of diffusely arranged polymorphonuclear infiltrates (HE 312′5X).

FIG. 6b shows a detail of extensive polymorphonuclear infiltrates separating the muscular bundles (HE 500X).

### EXAMPLES

The anti-inflammatory capacity of the product of Formula (I), wherein R is a potassium cation, was tested: 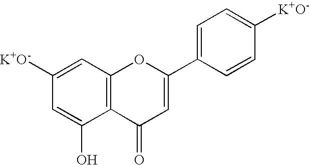
in male Swiss albino mice of 8 to 10 weeks of age (38-44 g), from the Servicio de Animales de Laboratorio (Laboratory Animals Department) of the University of Murcia. The animals were maintained under standard conditions (temperature of 22±2° C., humidity 60±4% and light/dark cycle of 12 hours) with food and drink ad libitum. The animals were anesthetized with ether in the moment of inducing inflammation and during the measurements of the volume of the paw.
Test of Paw Edema in Mice Induced by Carragenin

The inflammation was caused by intradermal injection of 0.05 ml of 1% Lambda Carrageenan (Sigma-Aldrich, Madrid, Spain) in saline solution in the plantar pad of the right hind paw. The measurement of the edema was carried out with a digital calibrator (Proinsa, Vitoria, Spain), measuring the 3 largest diameters of the paw for the volume calculation. The measurements were carried out in triplicate. The volume of the paw was measured immediately before (0 hours) and at 1, 5 and 24 hours after causing inflammation. The compound of Formula (I) was dissolved in sterile saline solution and intraperitoneally administered (70 mg/kg) 1 h before causing inflammation. The control group did not receive treatment. Groups of 10 animals were used.

The analysis of reliability of the measurement method used (digital electronic calibrator) has clearly shown that the measurements carried out in triplicate are significantly reliable (p<0.005).

Volume Evolution and Inflammation Inhibition

The intradermal injection of carragenin in the subplantar region of the paw in mice caused an increase of their volume from the first hour post-inoculation both in the control and in the treated group, although without showing significant differences with regard to 0 hours. The highest increase occurred at 5 hours in both groups, with significant differences with regard to 0 hours (p<0.005) and 1 hour post-inoculation (p<0.025). At 24 hours, the volume increase was lower, with significant differences both in the control and in the treated group, with regard to the values at 0 hours (p<0.005) and 1 hour post-inoculation (p<0.025) (FIG. 1).

The compound of Formula (I) was intraperitoneally administered at a dose of 70 mg/kg one hour before the intradermal injection of carragenin in the subplantar region of the paw. Increases in the volume of inoculated paw were lower in the treated group than in the control group, significant differences existing after one hour (p<0.025), at 5 hours (p<0.005) and at 24 hours (p<0.0005) (FIG. 2).

Microscopic Study and Assessment

The plantar pad area injected was extirpated at 24 hours post-inoculation; fixed in 10% buffered neutral formaldehyde, included in paraffin by the standard method, sectioned at 3 μm and stained with hematoxilin-eosin for its optical study which was carried out by two observers. The presence of edema and polynuclear neutrophils and monocytes was measured, being assessed as 0 (absence), 1 (slight), 2 (moderate) and 3 (intense) (FIGS. 3 to 6b).

The results of the microscopic study clearly show the anti-inflammatory activity of the treatment with 2,3-DHNA. At 24 hours in 60% of the samples of the treated group, neither the presence of edema nor inflammatory infiltrate was observed (degree 0); whereas 26.65% showed a slight inflammation (degree 1) and 13.3% moderate (degree 2). In the control group (untreated), 53.3% of the samples showed an intense inflammation (degree 3) and 46.7% moderate (degree 2).

Statistical Analysis

A two-way repeated measures analysis of variance, complemented with a two-by-two test of equality of means by means of the Student's t-test, was carried out.

For the analysis of the reliability of the measurement method used, the interclass correlation coefficient has been applied.

Immunochemical Assay: TNFα Measurement

For the immunochemical analyses, blood was extracted from the studied animals after 24 h of treatment, by means of cardiac puncture in Eppendorf tubes without heparin. It was maintained for 12 h at 2-8° C. and was centrifuged at 2000 g. Serum aliquots were taken and frozen at −80° C. The TNFα measurement was subsequently carried out using an immunoassay enzyme kit provided by the R&D Systems company (catalogue number MTA00).

The technique used is based on that of the quantitative sandwich enzyme-immunoassay. Microplates covered with an affinity-purified, polyclonal, anti-TNFα antibody were used. The test serum (in addition to known quantities of a TNFα standard for the calibration line, provided with the kit) is incubated in the microplates. The unbound material is eliminated by washing and the complex is again incubated with a new polyclonal, TNFα-specific covalently bound to peroxidase. The quantity of bound peroxidase is measured by reaction with a chromogenic substrate and is proportional to the quantity of TNFα present in the test serum sample.

The obtained TNFα values for the control animals and those treated with 2,3-DHNA (Table 1) confirm the results shown by the inflammation assay and by the microscopic study.

TABLE 1

| | TNFα (pmol/ml) |
|---|---|
| Control Group | 143.07 |
| Group treated with 2,3-DHNA | 54.89 |

Administration Method

For the intraperitoneal administration of 2,3-DHNA in humans, an injectable composition is designed, to assure the presence of the compound in the bloodstream, composed of: sterile saline solution, monopotassium phosphate, potassium hydroxide, propylene glycol and benzyl alcohol according to the description given in Example 1.

### Example 1.

TABLE 2

| Injectable composition | % by weight |
|---|---|
| 2,3-DHNA | 0.30% |
| Sterile saline solution | 88.55% |
| Potassium phosphate | 0.10% |
| Potassium hydroxide | 0.05% |
| Propylene glycol | 10.00% |
| Benzyl alcohol | 1.00% |

1. Use of compounds of formula 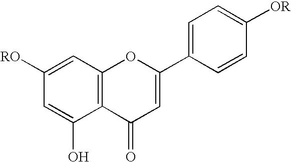
wherein the radicals R, independently from one another, are selected from among a hydrogen atom, a cation or a linear or branched C₁-C₃ alkyl group in the preparation of a drug for the treatment or prophylaxis of inflammatory processes and chronic diseases derived from inflammatory processes.

2. Use according to claim 1, wherein R is a cation of an alkaline metal.

3. Use according to claim 1, wherein R is a potassium cation.

4. Use according to claim 1, wherein the chronic diseases are selected between rheumatoid arthritis and Chrom's disease.

5. Use according to claim 1, wherein the inflammatory processes have the release of prostaglandins and thromboxanes as an origin.

6. An injectable pharmaceutical composition characterized in that it comprises a compound of formula 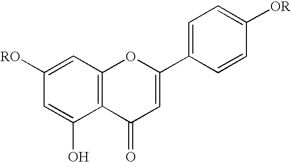
wherein the radicals R, independently from one another, are selected from among a hydrogen atom, a cation or a linear or branched C₁-C₃ alkyl group, saline solution, a glycol selected from among C₁-C₃ alcohols, an alkaline phosphate and an alkaline hydroxide.

7. An injectable pharmaceutical composition according to claim 6, wherein R is a cation of an alkaline metal.

8. A pharmaceutical composition according to claim 6, wherein R is a potassium cation.

9. A pharmaceutical composition according to claim 6, wherein the glycol is propylene glycol.

10. A pharmaceutical composition according to claim 6, wherein the alkaline phosphate is potassium phosphate.

11. A pharmaceutical composition according to claim 6, wherein the alkaline hydroxide is potassium hydroxide.
